(19) United States
(12) Patent Application Publication
(10) Pub. No.: US 2005/0251193 A1
(43) Pub. Date: Nov. 10, 2005

(54) Combination catheter and stent system

(76) Inventor: G. Banning Lary, Miami, FL (US)

Correspondence Address: MCHALE & SLAVIN, P.A., 2855 PGA BLVD, PALM BEACH GARDENS, FL, 33410, US

(21) Appl. No.: 10/889,738

(22) Filed: Jul. 13, 2004

Related U.S. Application Data
(60) Provisional application No. 60/498,974, filed on Aug. 29, 2003.
(60) Provisional application No. 60/528,579, filed on Dec. 9, 2003.
(60) Provisional application No. 60/529,670, filed on Dec. 15, 2003.

(51) Int. Cl. A61M 29/00

(52) U.S. Cl. 606/192

(57) ABSTRACT

A combination catheter system for improving flow in an occluded body passageway includes a guide wire for proper placement of a catheter. The catheter has radial blades mounted on a component shaped to expand the occlusion. A stent is removably mounted on a second component which places and expands the stent for reinforcing the passageway.

### RELATED APPLICATIONS

This application claims the benefit of the earlier filing date of Provisional Applications 60/498,974, filed Aug. 29, 2003; 60/528,579, filed Dec. 9, 2003; and 60/529,670, filed Dec. 15, 2003 under 35 USC 119(e).

### FIELD OF THE INVENTION

This invention relates to the field of surgical catheters for reducing stenosis and placing stents in blood vessels.

### BACKGROUND OF THE INVENTION

Stenotic and occlusive vascular disease of the arteries can eventuate in death of tissues of many systems, especially the intestines, lower extremities, liver, kidneys and heart. Any significant reduction or restriction in the flow of blood through the arteries of the body can cause complications which may have serious ischemic consequences. Arterial blockages caused by plaque and fibrotic stenosis in coronary arteries are known to be a leading cause of heart attacks, subsequent strokes, and other debilitating maladies. In recent years, the transdermal cannulation of major arteries supplying these organ systems has permitted a direct attack on occlusive and stenotic arterial disease. Dilation, incision, atherectomy, and stent placement have simplified procedures and returned improved results. Further, balloon catheters are now used in other passageways or ducts in the body, as are stents, eg., the urethra and other tubular passageways.

Angioplasty is a conventional procedure for treating occlusions or stenosis in the major vessels of the heart. Normally, a balloon catheter is inserted into the vessel with the balloon positioned in the occluded area. The balloon is inflated or otherwise expanded to compress the occluding material or plaque into the vessel wall. The plaque remains in the artery and is not removed. Unfortunately, in some cases, it appears that the plaque which remains in the artery may still present a stenosis. Furthermore, in approximately 30-60% of the vessels treated by angioplasty, there is a restenosis. This high recurrence rate is thought to be the result of fibrotic contraction in the lumen of the vessel.

Improved results have been gained by having an expandible wire stent encircling the deflated balloon. The inflation of the balloon expands the stent and permanently fixes it in the wall of the vessel to reinforce the vessel and prevent re-occlusion after the balloon is removed. However, the stent, as well as the balloon, adds stiffness to the shaft of the catheter reducing the ability of the catheter to follow a tortuous path through partial occlusions in a vessel.

Treatment methods may include percutaneous, intraluminal installation of one or more expandable, tubular stents or prostheses in sclerotic lesions. Stents or prostheses function to maintain patency of a body lumen and especially to such implants for use in blood vessels. The stents are typically formed from a cylindrical metal mesh which expand when internal pressure is applied. Alternatively, they can be formed of wire wrapped into a cylindrical shape. Stents or prostheses can be used in a variety of tubular structures in the body including, but not limited to, arteries and veins, ureters, common bile ducts, and the like. Stents are used to expand a vascular lumen or to maintain its patency after angioplasty or atherectomy procedures, overlie an aortic dissecting aneurysm, tack dissections to the vessel wall, eliminate the risk of occlusion caused by flaps resulting from the intimal tears associated with primary interventional procedure, or prevent elastic recoil of the vessel.

These metallic stents are deployed inside an arterial segment and embedded in the vessel to maintain patency typically after angioplasty or atherectomy interventions. Once they are so positioned, they are extremely difficult to remove. Often the vessels in which they are placed become occluded or severely restenosed in a relative short period of time. These complications continue to occur the longer the stents remain in place, resulting in total or partial obstruction of blood flow through the artery. Usually, the distal portion of the artery will remain patent and is supplied by collateral circulation through branches of other major arteries. However, tile decreased direct blood flow results in many cardiac problems.

It has also been shown that when an angioplasty procedure is performed after the stenotic segment is longitudinally incised, the opening established through the segment is much larger as compared to standard angioplasty without the prior incisions. Still further, the increase in the opening in the stenotic segment is accomplished without tearing the vessel wall. Moreover, it has been found that incising the stenosis prior to dilation allows greater compression of the stenotic tissue with decreased likelihood of the stenosis rebuilding at a later date. As those skilled in the art will appreciate, the plaque creating a common arterial stenosis is somewhat fibrous and will tend to return to its original predilation configuration. With this fibrous composition, the stenosis is therefore more likely to maintain a compressed configuration if the fibers are incised prior to balloon dilation. On the other hand, if the fibers in the stenosis is not incised first, the completeness of the compression of the stenosis is dependent on whether the inflated balloon is able to break apart fibers in the tissue as those skilled in the art will recognize, dilation of a segment is of course limited by the arteries able to withstand dilation. Over-dilation can have the catastrophic result of rupturing the vessel.

Several modifications of this conventional methodology have been patented, including U. S. Patent No. 6,726,677 to Flaherty et al which teaches a catheter with a tissue penetrator for punching through a vessel wall. Radisch, Jr., U.S. Pat. No. 6,632,231, which teaches the use of cutting blades on the exterior of the balloon for incising a stenosis to create a uniform surface. Deaton et al, U.S. Pat. No. 6,565,583, teaches a flexible blade cutter for longitudinally separating the plaque from the wall of the vessel for removal. Azizi, U.S. Pat. No. 6,558,401, teaches the use of two expanding members. A forward member for occluding the vessel to prevent particles entering the circulation system and an angioplasty balloon for expanding the stenosis. Lary, U.S. Pat. No. 6,306,151, teaches the use of a balloon catheter with a reciprocating blade for cutting the struts of a stent prior to removal. Lary, U.S. Pat. No. 6,258,108, teaches a tapered balloon with blades on the tapered surface.

The prior art devices give good results in cases where the stenosis and obstruction is well defined and segmental. However, there are cases in which the stenosis is ill defined, irregular or elongated with multiple obstructions. Also, there may be multiple stenotic areas in the same vessel. What the prior art lacks is a surgical catheter that is capable of incising plaque, carrying a stent and having flexibility in the leading end for maneuverability.

### SUMMARY OF THE PRESENT INVENTION

Disclosed is a combination catheter system for improving flow in a passageway having an occlusion. The catheter system has an elongated catheter shaft suitable for advancement through an internal passageway of the body. The catheter shaft defined by a leading end and a trailing end with multiple lumen extending from the leading end to the trailing end. A first inflatable balloon means is disposed near the leading end for incising the occlusion, the balloon means having a plurality of blades extending radially outwardly. A stent is spaced from the balloon means toward the trailing end with a second balloon expanding means fixed to the shaft for expanding the stent. The stent encircling the second balloon. One of the the multiple lumens is adapted to communicate with the expanding balloon means whereby the shaft is advanced until the occlusion is incised and the stent is positioned in the incised occlusion. The second balloon means is activated through the one lumen to expand the stent in the incised occlusion to improve flow.

The incisions destroy the continuity of the surface of the stenosis thereby reducing the pressure required to compress the plaque. As the balloon is inflated, the depth of the incisions increase and the plaque is sub-divided into sections further contributing to the reduction of the occlusion.

Therefore, it is an objective of this invention to provide a combination catheter system having multiple functions of incising, dilating, irrigation, and stent placement.

It is another objective of this invention to provide a combination catheter system with the customary longitudinal cutting blades on the surface of a balloon, or with short cutting blades attached to the forward (distal) body of a balloon and a stent spaced apart along the shaft of the catheter.

It is a further objective of this invention to provide a combination catheter with a flexible shaft portion separating the balloon and the stent.

It is yet another objective of this invention to provide a combination catheter system for improving flow in ducts having multiple occluded areas.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

FIG. 1 is a longitudinal cross section of a vessel and a catheter system of this invention disposed therein;

FIG. 2 is a longitudinal cross section of a vessel with a tortuous passageway with a catheter system of this invention disposed therein;

FIG. 3 is a longitudinal cross section of the vessel of FIG. 2 with a stent in situ;

FIG. 4 is a longitudinal cross section of a vessel with a modified incising catheter of this invention in situ;

FIG. 5 is a longitudinal cross section of a vessel with a catheter system of this invention in situ with a collapsed balloon and stent;

FIG. 6 is a longitudinal cross section of a vessel with a catheter system of FIG. 5 expanded in situ; and

FIG. 7 is a longitudinal cross section of a vessel with a catheter system of this invention in situ including an irrigation catheter.

### DETAILED DESCRIPTION OF THE INVENTION

As shown in FIG. 1, a guide wire 20 is inserted into the vessel 21 and extended through the stenotic area 22. The guide wire 20 may have a radiopaque marker 23 at the leading end for locating the guide wire in reference to the stenosis. Once the guide wire is properly positioned, the catheter 10 is telescoped over the trailing end of the guide wire and follows the guide wire to the stenosis. The catheter 10 has a multi-lumen shaft 11 with a lumen 12 for expanding the leading end balloon 13.

As shown, the balloon 13 has several exterior blades 14, 15 extending radially from the balloon. Once the balloon 13 is placed to transgress the length of the stenosis it is expanded to deploy the blades 14, 15. Alternately, the small dilating balloon 53 with cutting blades on the forward or distal end is distended and passes through and dilates the stenosis by a reciprocating action. The catheter may be manipulated so that the balloon is positioned within the stenotic area and expanded in place. The incisions destroy the continuity of the surface of the stenosis thereby reducing the pressure required to compress the plaque. As the balloon 13 is inflated, the depth of the incisions increase and the plaque is sub-divided into sections further contributing to the reduction of the occlusion.

A second expandable balloon 16 is spaced along the shaft 11 of the catheter further from the leading end. The multi-lumen shaft has another lumen 17 for expanding the second balloon 16. A collapsed stent 18 encircles the balloon 16. After the surgical balloon 13 transits the stenosis and incises and compresses the occlusion, the catheter 10 is advanced to place the balloon 16 and stent 18 in the stenosis. The second balloon 16 is expanded to enlarge the stent and lock the struts 19 in the expanded position. The balloon 16 is then collapsed leaving the stent 18 as a reinforcement in the vessel 21. The catheter 10 with the collapsed balloons 13 and 16 is then withdrawn from the vessel completing the procedure.

In FIG. 1, the leading end balloon 13 has an elongated body with approximately constant diameter and the blades 14, 15 oriented longitudinally and spaced about the circumference. The catheter may be manipulated to reciprocate through the stenotic area, if desired.

Between the surgical balloon 13 and the second balloon 16, there is a more flexible portion 40 of the shaft of the catheter with an inner coil 41 maintaining the diameter of the bore while permitting longitudinal bending of the shaft 11. The coil 41 may be formed between the inner and outer walls of the catheter shaft or on either the inside or outside thereof. Because the coil 41 provides an open bore, the lumen in the multi-lumen shaft are not blocked as the catheter follows a tortuous path through a partially occluded vessel, as illustrated in FIG. 2.

In FIG. 2, the catheter 30 has a surgical balloon 33 with an elliptical leading end. A guide wire extends, at least, through the leading end of the catheter. Several blades 34, 35 are attached to the elliptical surface about the circumference. These blades result in a gradually enlarged opening as the balloon 33 advances through an occluded area. A second balloon 36 is spaced rearwardly of the first balloon and carries a collapsed stent 38. After the stenosis has been reduced, the catheter 30 is advanced to move the stent 38 into the occluded area. The balloon 36 is expanded locking the stent 38 in the walls of the vessel. The balloon is collapsed to free the stent.

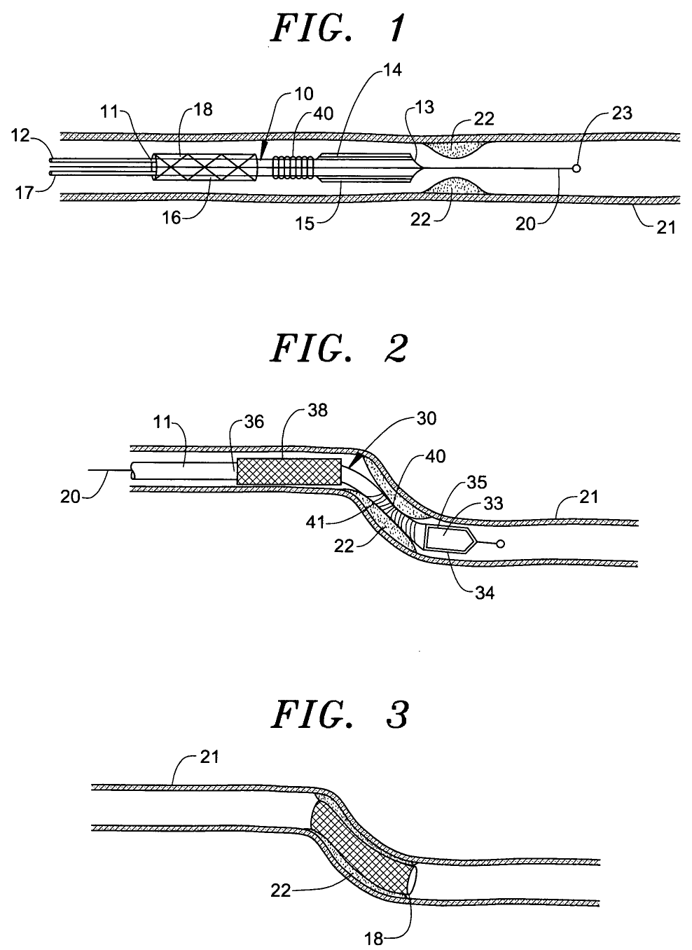

After the stent 38 is in place and both balloons are collapsed, the catheter is withdrawn through the stent and the vessel. The stent is embedded in the wall of the vessel, as shown in FIG. 3.

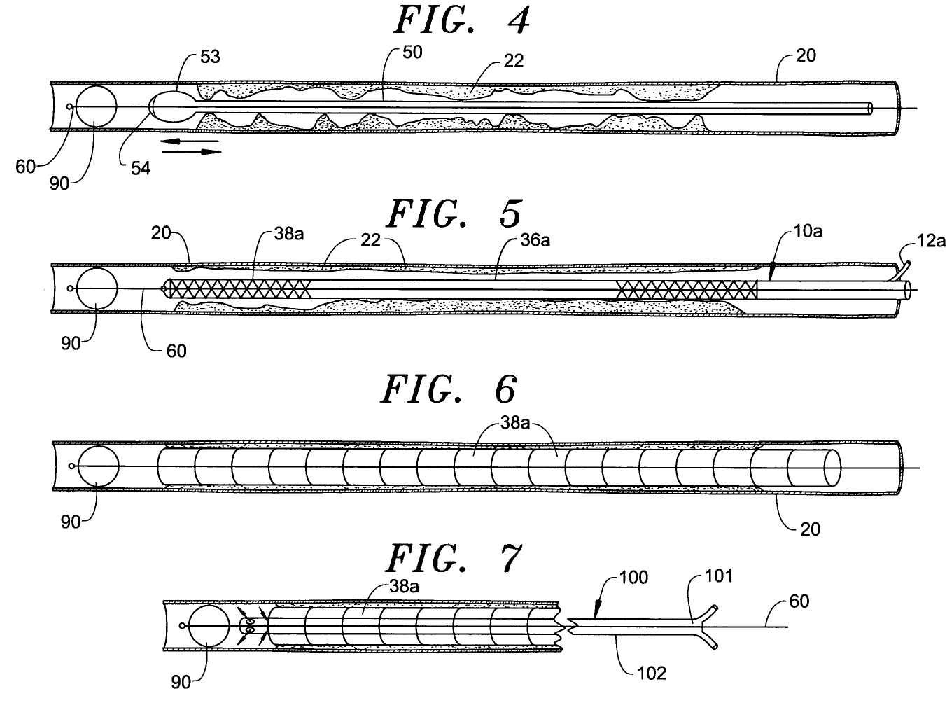

FIGS. 4-7 are a representation of a tubular conduit in the body with an elongated stenosis creating a tortuous flow path. The catheter system described above, in reference to FIGS. 1-3, can be used in this situation, merely requiring that the balloons and stent be made of a length necessary to extend through the length of the affected area.

However, the incising function of the catheter may be accomplished by a modification of the incising balloon. In FIG. 4, a solid ovoid 53 is connected to the leading end of a catheter 50 with an arcuate incising blade 54. The catheter 50 may be separately advanced over the guide wire 60 or telescoped within catheter until approximate to the occluded area. The guide wire 60 extends through the leading end of the catheter 50 and terminates with an occluding balloon 90. The guide wire 60, with the occluding balloon 90, is passed through the stenosis and expanded beyond the stenosis to prevent plaque from entering the system.

The catheter 50 is advanced through the stenotic area to a position near the occluding balloon 90. Longitudinal incisions in the stenotic segment are made by blades 54 attached to the distal end of the balloon 53. These blades are exposed by distending the balloon. The blades do not research the diameter or the balloon so that the apparatus cuts and dilates the stenosis without perforating the wall of the vessel. The incising blades may be used with a single pass or by a reciprocating action to transgress the stenosis. The catheter 50 is then withdrawn along the guide wire 60.

The multi-lumen catheter 10a is advanced over the guide wire 60 through the stenotic area, as shown in FIG. 5. The balloon 36a is fixed to the shaft of the catheter and stent 38a surrounds the collapsed balloon. Once the catheter is correctly positioned, the balloon 36a is expanded to extend the struts of the stent 38a. The balloon 36a is collapsed leaving the stent 38a in place, as shown in FIG. 6.

As shown in FIG. 7, the balloon catheter 10a is withdrawn and an irrigation catheter 100 may be advanced over the guide wire 60 toward the occluding balloon 90. The irrigation catheter 100 has a inflow line 101 and an exhaust line 102. Irrigation fluid is introduced in the inflow line and the area between the stent 38a and the occluding balloon 90 is perfused to remove any plaque through the exhaust line 102. The irrigation catheter 100 is then removed. The occluding balloon is collapsed and the guide wire is removed.

A number of embodiments of the present invention have been described. Nevertheless, it will be understood that various modifications may be made without departing from the spirit and scope of the invention. Accordingly, it is to be understood that the invention is not to be limited by the specific illustrated embodiment but only by the scope of the appended claims.

1. A combination catheter system for improving flow in a passageway having an occlusion comprising an elongated catheter shaft suitable for advancement through an internal passageway of the body, said shaft having a leading end and a trailing end with multiple lumen extending from said leading end to said trailing end, a first means near said leading end for incising the occlusion, a stent spaced from said first means toward said trailing end, an expanding means fixed to said shaft for expanding said stent, said stent encircling said expanding means, one of said multiple lumen adapted to communicate with said expanding means whereby the shaft is advanced until the occlusion is incised and the stent is positioned in the incised occlusion, said expanding means activated through said one lumen to expand said stent in the incised occlusion to improve flow.

2. A combination catheter system of claim 1 wherein said first means is shaped as an oval, a plurality of blades extending radially outwardly from said oval, said blades oriented longitudinally along said oval.

3. A combination catheter system of claim 1 wherein said first means is an expandable balloon, a plurality of blades extending radially from the surface of said expanded balloon and oriented longitudinally of said balloon.

4. A combination catheter system of claim 3 wherein said first means is an expandable balloon, said balloon being ovoid in shape.

5. A combination catheter system of claim 3 wherein said first means is an expandable balloon, said balloon being tubular in shape.

6. A combination catheter system for improving flow in a passageway having an occlusion comprising an elongated catheter shaft suitable for advancement through an internal passageway of the body, said shaft having a leading end, a trailing end and multi-lumen, a first balloon near said leading end of said shaft, a second balloon separated from said first balloon by a space along said shaft, said first balloon adapted to enlarge the occlusion, a stent removably surrounding said second balloon, said second balloon adapted to expand said stent in the occlusion, said stent adapted to remain and reinforce said passageway, said space between said first and second balloon having greater flexibility than said first balloon and said second balloon whereby said shaft can follow a tortuous path.

7. A combination catheter system of claim 6 wherein said first balloon has a plurality of blades extending radially therefrom, said blades oriented longitudinally of said first balloon whereby said blades incise the occlusion when said balloon is expanded.

8. A combination catheter system of claim 6 wherein said first balloon is oval in shape.

9. A combination catheter system of claim 6 wherein said first balloon is tubular in shape.

10. A combination catheter system of claim 6 wherein a guide wire is slidably disposed in a first lumen of said multi-lumen, said first balloon expanded by a second lumen and said second balloon expanded by a third lumen.

11. A combination catheter system of claim 6 wherein said guide wire has a leading end and a trailing end, an occluding balloon attached to said leading end.

12. A combination catheter system for improving flow in a body passageway having an occlusion comprising an elongated guide wire with a leading end and a trailing end, an occluding balloon connected to said leading end, an elongated catheter with a multi-lumen shaft adapted to telescope over said guide wire, said catheter having an incising means and an expanding means, a removable stent encircling said expanding means, an irrigating catheter having an inflow channel and an exhaust channel whereby said guide wire is advanced through the occlusion, said occluding balloon is expanded to block the passageway, said catheter is advanced along said guide wire through the occlusion, incising the occlusion and said stent is placed in the incised occlusion, said expanding means expands said stent to reinforce the passageway, said catheter being withdrawn leaving said stent in place, said irrigating catheter advanced along said guide wire through said stent toward said occluding balloon and said passageway being perfused.
